(19) United States
(12) Patent Application Publication
(10) Pub. No.: US 2016/0030729 A1
(43) Pub. Date: Feb. 4, 2016

(54) LUER CONNECTOR

(71) Applicant: ULTRADENT PRODUCTS, INC., South Jordan, UT (US)

(72) Inventor: Bruce S. McLean, Sandy, UT (US)

(21) Appl. No.: 14/775,319

(22) PCT Filed: Mar. 12, 2014
(86) PCT No.: PCT/US14/23973

§ 371 Date: Sep. 11, 2015

Related U.S. Application Data
(60) Provisional application No. 61/778,172, filed on Mar. 12, 2013.

(51) Int. Cl. A61M 39/10

(52) U.S. Cl. CPC A61M 39/1011; A61M 2039/1033; A61M 2039/1038; A61M 2039/1094; A61M 5/345

(57) ABSTRACT

The present disclosure is drawn to a device for injecting or extracting fluid, comprising a channeling body having a distal end and a proximal end, wherein the channeling body is adapted for injecting or extracting fluid through an opening at the distal end. The device also includes a male luer connector at the distal end about the opening, and can further include an arm attached to the channeling body. The male luer connector can be adapted to engage with a female luer connector. The arm can also be configured to lock the female luer connector in place along an outer surface of the female luer connector.

### BACKGROUND

Male and female luer connectors have been used to join various fluidic devices together. Typically, a luer connector can be threaded along an outer surface on one fluidic device, and the other luer connector can have matching threads on an inner surface of a luer skirt on another fluidic device. Thus, when the male luer connector and the female luer are pressed together and twisted in opposing directions, the opposing luers connectors become tightly connected in a luer lock configuration. This is a typical arrangement for connecting fluid injection or extraction devices. With this in mind, it would be advantageous to provide other mechanisms for connecting fluid injection or extraction devices that are simple to manufacture, and which provide an acceptable degree of locking for a given application.

### SUMMARY

The present disclosure is drawn to a device for injecting or extracting fluid, comprising a channeling body having a distal end and a proximal end, wherein the channeling body is adapted for injecting or extracting fluid through an opening at the distal end. The device also includes a male luer connector at the distal end about the opening, and can further include an arm attached to the channeling body, wherein the male luer connector is adapted to engage with a female luer connector. The arm can also be configured to lock the female luer connector in place along an outer surface of the female luer connector.

In another embodiment, a method of assembling a device for injecting or extracting fluid can include the step of obtaining a channeling body for injecting or extracting fluid through an opening at a distal end. The channeling body can include a proximal end and a distal end with a male luer connector at the distal end about the opening. The male luer connector can also comprise an arm attached to the channeling body. The method can also include the steps of obtaining an adaptor comprising a female luer connector, and connecting the female luer connector to the male luer connector such that the arm locks the female luer connector in place along an outer surface of the female luer connector.

In another example, a modular system for assembling a device can comprise an adaptor including a female luer connector. The modular device can also include a channeling body having a distal end and a proximal end, wherein the channeling body is configured for injecting or extracting fluid through an opening thereof. The channeling body can include a male luer connector at the distal end about the opening, and the male luer connector can comprise an arm attached to the channeling body. The male luer connector can be adapted to connect to the female luer connector such that the arm locks the female luer connector at a location along an outer surface of the female luer connector.

### BRIEF DESCRIPTION OF THE DRAWINGS

FIG. 1 is a perspective view of a syringe including a male luer connector as well as two alternative needle adaptor tips in accordance with examples of the present disclosure;

FIG. 2 is a perspective view of a suction tube for fluidly connecting an extractor or ejector to a suction tip;

FIG. 3 is a perspective view of a suction tube with a male luer connector for fluidly connecting an extractor or ejector to a suction tip in accordance with examples of the present disclosure;

FIG. 4 is an alternative perspective view of a suction tube with a male luer connector for fluidly connecting an extractor or ejector to a suction tip in accordance with examples of the present disclosure;

FIGS. 5 and 6 present a perspective view and a cross-section view, respectively, of a male luer connector at a distal end of a channeling body, such as a syringe barrel or a suction tube, in accordance with examples of the present disclosure;

FIG. 7 is a side view of a male luer connector at a distal end of a channeling body, such as a syringe barrel or a suction tube, in accordance with examples of the present disclosure;

FIG. 8 is a side view of a male luer connector at a distal end of a channeling body, such as a syringe barrel or a suction tube, in accordance with examples of the present disclosure;

FIG. 9 is a side view of a male luer connector at a distal end of a channeling body, such as a syringe barrel or a suction tube, in accordance with examples of the present disclosure;

FIG. 10 is a side view of a male luer connector at a distal end of a channeling body, such as a syringe barrel or a suction tube, in accordance with examples of the present disclosure;

FIG. 11 is a side view of a male luer connector at a distal end of a channeling body, such as a syringe barrel or a suction tube, in accordance with examples of the present disclosure; and

FIG. 12 is a bottom view of the male luer connector of FIG. 11.

### DETAILED DESCRIPTION

Before the present invention is disclosed and described, it is to be understood that this disclosure is not limited to the particular process steps and materials disclosed herein because such process steps and materials may vary somewhat. It is also to be understood that the terminology used herein is used for the purpose of describing particular examples only. The terms are not intended to be limiting because the scope of the present disclosure is intended to be limited only by the appended claims and equivalents thereof.

It is noted that, as used in this specification and the appended claims, the singular forms “a,” “an,” and “the” include plural referents unless the context clearly dictates otherwise.

The term “channeling body” refers to any structure that can be used to channel a fluid, including structures such as syringe barrels, suction tubes, medical tubing such as for intravenous injections or drainage, or the like. The channeling body, in accordance with examples of the present disclosure, includes a male luer connector as described herein.

As used herein, the term “angled” when referring to the male luer connector refers to the relative angle of the male portion of the connector relative to a longitudinal axis of the channeling body of the device, e.g., syringe barrel, suction tube, etc.

The term “medical” or “medical device” as used herein includes any system, device, or method that is used for the diagnosis or treatment of a subject, including a human or other subject. Thus, the term “medical” is inclusive of any procedure or device used in laboratories, medicine, dentistry, veterinarian disciplines, or the like.

The term “fluid” refers to any liquid or fluid suspension that can be mechanically injected or extracted with the devices of the present disclosure.

The term “adaptor” is used generally to describe the various tips, caps, syringes, or other structures that can be connected to the male luer connectors of the present disclosure. Exemplary adaptors can include injection needles, suction tips, dispensing brush tips, or the like. Other adaptor structures that can be used include luer caps, syringes, or the like.

The term “substantially” provides a small degree of flexibility in interpretation of the meaning of a term. For example, the term “substantially flattened” would include tubular structures that are flattened with respect to a typical circular cross-section.

As used herein, a plurality of items, structural elements, compositional components, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Dimensions, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3.5, and 4 and sub-ranges such as from 1-3, from 2-4, and from 3-5, etc. This same principle applies to ranges reciting only one numerical value. Furthermore, such an interpretation should apply regardless of the breadth of the range or the characteristics being described.

With these definitions in mind, the present disclosure is drawn to a device for injecting or extracting fluid, comprising a channeling body having a distal end and a proximal end, wherein the channeling body is adapted for injecting or extracting fluid through an opening at the distal end. The device also includes a male luer connector at the distal end about the opening, and can further include an arm attached to the channeling body, wherein the male luer connector is adapted to engage with a female luer connector. The arm can also be configured to lock the female luer connector in place along an outer surface of the female luer connector.

In another embodiment, a method of assembling a device for injecting or extracting fluid can include the step of obtaining a channeling body for injecting or extracting fluid through an opening at a distal end. The channeling body can include a proximal end and a distal end with a male luer connector at the distal end about the opening. The male luer connector can also comprise an arm attached to the channeling body. The method can also include the steps of obtaining an adaptor comprising a female luer connector, and connecting the female luer connector to the male luer connector such that the arm locks the female luer connector in place along an outer surface of the female luer connector.

In another example, a modular system for assembling a device can comprise an adaptor including a female luer connector. The modular device can also include a channeling body having a distal end and a proximal end, wherein the channeling body is configured for injecting or extracting fluid through an opening thereof. The channeling body can include a male luer connector at the distal end about the opening, and the male luer connector can comprise an arm attached to the channeling body. The male luer connector can be adapted to connect to the female luer connector such that the arm locks the female luer connector at a location along an outer surface of the female luer connector.

In each of the various embodiments herein, whether discussing the device, the modular system, or the method, there may be some common features of each of these embodiments that further characterize options in accordance with principles discussed herein. Thus, any discussion of the device, or related systems or methods, either alone or in combination, is also applicable to the other embodiments not specifically mentioned. For example, a discussion of the male luer connector in the context of the device is also applicable to the related methods or systems, and vice versa.

In each of these example embodiments, it is noted that there are many different types of channeling bodies that can be appropriate for use. For example, the channeling body can be a barrel of a syringe, a suction tube configured for use with a suction extractor or ejector, e.g., High Volume Extractor (HVE) or Saliva Ejector (SE), an elongated flexible tube for intravenous injection infusion, a drainage tube, etc. Thus, discussion of a specific type of channeling body is applicable to other types of channeling bodies, whether mentioned specifically or not. Also, as mentioned, at a distal end of the channeling body is a male luer connector that includes an arm that locks the female luer connector in place from an outer surface thereof. It is noted, however, that multiple arms can likewise be used, e.g., 2, 3, 4, etc. Thus, the male luer connector can consist of a single arm, can consist of two arms, or can comprise two (or more) arms. Because there is not typically a threaded skirt that surrounds the male portion of the luer lock, in some examples, the male luer locks can be said to be skirtless.

In another example, the arm can also comprise an engagement protrusion that is part of the arm, but which protrudes outwardly from the arm (toward the male portion of the male luer connector). In this example, the protrusion can be positioned and adapted to lock the female luer connector in place by engaging with a recess on the outer portion of the female luer connector. The recess on the female luer connector can be a recessed groove or hole, or can be provided by treads, for example. Alternatively, the female luer lock can include a raised ridge that is in place for the engagement protrusions to rest beyond to provide a locking function similar to that provided by the recessed groove.

Whether there is a single arm, or multiple arms, each arm may have a protrusion and/or multiple protrusions. Furthermore, in one specific example, the male luer connector can comprise a first arm including a first engagement protrusion and a second arm including a second engagement protrusion. In one example, the first engagement protrusion on the first arm can be misaligned with respect to the second engagement protrusion on the second arm. This may be useful with the recesses on the female luer connector are provided by threads, and the threads at the location of the protrusions are at different elevation relative to the axis of the arm. In another example, the first engagement protrusion on the first arm can be aligned with respect to the second engagement protrusion on the second arm.

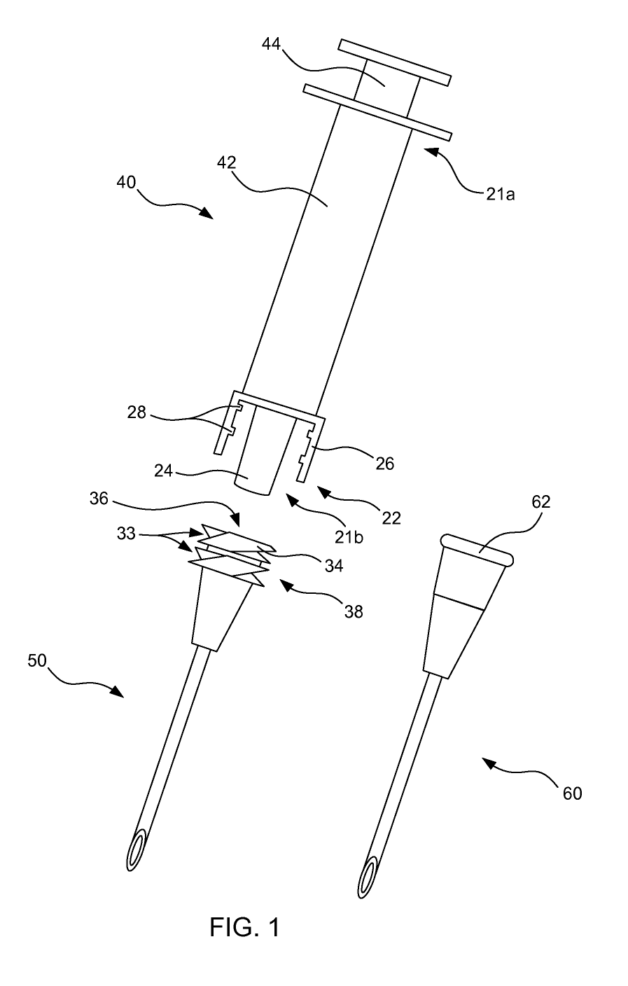

Turning now to the FIGS., FIG. 1 is a perspective view of a syringe 40 including a male luer connector 22 and a needle 50 with and a female luer connector 38 in accordance with an embodiment of the present disclosure. Thus, in this example, the channeling body is a barrel 42 of the syringe having a proximal end 21a and a distal end 21b. A plunger 44 is likewise shown which is used for pushing or pulling fluid through the barrel and the opening at the distal end of the channeling body (which in this case is at the distal end of the male portion 24). In this example, the male luer connector includes a pair of arms 26, and each arm includes a pair of engagement protrusions 28. Because arms are used to interface with the female luer connector and there is no fully surrounding skirt present, this arrangement can be said to be a skirtless luer lock. In this specific example, the protrusions on the respective arms can be misaligned with one another, as illustrated by the dashed line. Thus, unlike other examples where the protrusions are aligned with one another, misalignment can be used for specific female luer connector arrangements. However, the protrusions can likewise be aligned, as shown in subsequent FIGS. hereinafter. The needle, as shown, or other adaptor, can be connected to the male luer connector by twisting the female luer connector so that recesses 33 of the threads 34 are engaged with the engagement protrusions of the male luer connector. Thus, the male portion of the male luer connector can be inserted into the female portion 36 of the female luer connector, and the two luer connectors can be locked together accordingly. Though a needle is specifically shown, other adaptor tips can likewise be used, such as a suction tip, a cap, a syringe adaptor, or another syringe.

Also shown in FIG. 1 is an alternative adaptor tip, shown at 60, that utilizes a different type of female luer connector. Specifically, the adaptor tip in this example includes a ridge 62, rather than threads or recess shown at 33. In this example, the engagement protrusions of the male luer connector can interlock with the ridge as the protrusions are pressed just past the ridge. The arms in this example can flex slightly to allow the protrusions to pass the ridge and lock in place just beyond the ridge. Alternatively, the ridge can include a threaded portion so that the engagement protrusions can pass by the ridge and connect just beyond the ridge.

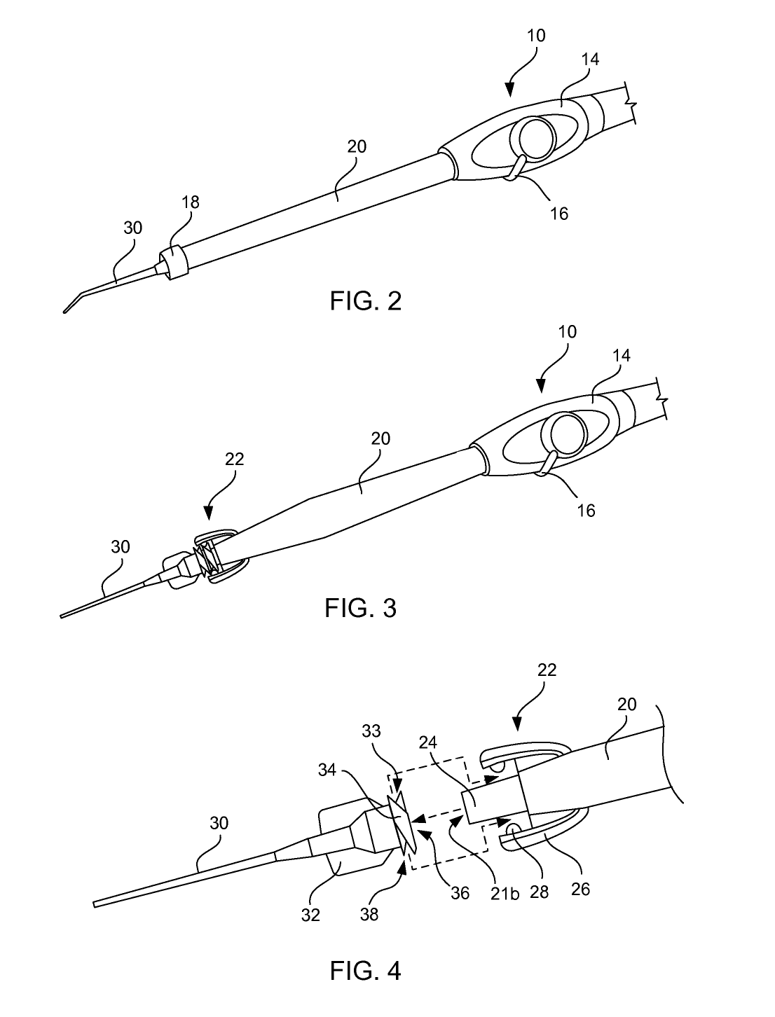

FIG. 2 is a perspective view of a suction tube 20 for fluidly connecting an extractor or ejector 10 to an adaptor, which in this case is a suction tip 30. In this embodiment, the extractor or ejector can be a High Volume Evacuator (HVE) or a Saliva Ejector (SE), or any other suction device used in the medical arts, including dentistry. The extractor or ejector includes a handle 14 and a switch 16 which controls a shut-off valve (not shown). The extractor or ejector is shown as being directly fluidly coupled to the suction tube, though if the suction tube is not appropriately sized for use with this particular extractor or ejector, a step-up or step-down coupler (not shown) may be used. For example, it is common for a suction tube to have an outer radius size that is appropriately sized to fit within a Saliva Ejector (SE), but is too small for a High Volume Evacuator (HVE), and thus, a step-down coupler is used so that the two pieces of equipment can be fitted appropriately together. In further detail, the suction tube in this example includes a fully skirted luer connector 18. Furthermore, a suction tip 30 is also shown that can be used in endodontics for cleaning fluid and necrotic material out of a tooth that has undergone a root canal procedure prior to filling the space with fill material. Thus, this fully skirted luer connector surrounds the interface where the suction tube is locked together with the suction tip or other adaptor tip.

FIG. 3 is a perspective view of a suction tube 20 for fluidly connecting an extractor or ejector 10 to a suction tip 30 in accordance with examples of the present disclosure. In this example, the extractor or ejector has the same features as that described in FIG. 2, including a handle 14 and a switch 16 for controlling a shut-off valve (not shown). For example, a High Volume Extractor

(HVE) or a Saliva Ejector (SE) can be used to extract fluid in a dental environment. Normally, fluids such as water, saliva, and other compounds introduced in the mouth by a dentist can be removed. However, it is noted that these devices can likewise be used to extract solids, such as necrotic tissue, cement, metal particles, pieces of tooth or pulp, or the like.

The suction tube 30 in this example is different than the one described in FIG. 2. Specifically, in this example, the suction tube is flattened (compared to the substantially round diameter suction tube shown in FIG. 2), and more significantly, the luer connector is a male luer connector 22 prepared in accordance with examples of the present disclosure and described more fully in FIG. 4.

FIG. 4 provides a perspective view of a similar, but slightly modified device compared to FIG. 3. Specifically, only a single row of threads 34 is present on the suction tip 30 in this example. The device includes a suction tube 20 for fluidly connecting an extractor or ejector (not shown in this FIG., but shown in FIGS. 2 and 3) to the suction tip via a male luer connector 22. The male luer connector includes a male portion 24 for engaging with a female portion 36 of a female luer connector 38. The male luer connector in this example also includes a pair of support arms 26, each having an engagement protrusion 28 adapted to engage with and lock in place the threads of the female luer connector at recesses 33 found on the threads, or alternatively, lock in pace beyond the threads. In this example, the suction tip also includes a pair of flanges 32, which assists the user in twisting the suction tip and engaging the threads with the engagement protrusions. It is also noted that the male luer connector is positioned at or near a distal end 21b of the suction tube.

It is noted that FIG. 4 shows a suction tube 20, which is a single specific example of a channeling body of the present disclosure. Other channeling bodies, such as a barrel of a syringe, can also be acceptable for use in other examples of the present disclosure. Furthermore, it is noted that the suction tip 30 shown is one that can be usable with an extractor or ejector. However, the suction tip is merely one example of an adaptor that can be used. In other embodiments, the adaptor tip may be a needle or another type of tip suitable for use with the specific channeling body selected for use.

In further detail regarding the use of these devices with an HVE or an SE, several details can be useful in forming certain examples embodiments of the suction tubes or the suction tips described herein. For example, suction tips having an opening at the distal end thereof (the working end that is used to contact the tooth) can range from about 0.005 inch to about 0.1 inch. Other example ranges can be from about 0.01 inch to about 0.05 inch, or from about 0.015 inch to about 0.02 inch. Two specific example tips that can be used particularly in endodontics for cleaning fluid or necrotic tissue out of a drilled out tooth can be from about 0.014 inch or about 0.019 inch. Furthermore, regarding the suction tube, in one example, the male luer lock can be integrally molded with the tubular channel so that the suction tube (including the male luer lock) is a single piece. Furthermore, the suction tube can be constructed of a material and at a wall thickness so that it is stiff enough to act as a cheek retraction device. Thus, the dentist can retract the cheek easily with the suction tube without fear of the adaptor bending or creasing during use. Another feature that can be implemented is to utilize a material for the suction tube that is transparent or at least translucent. Thus, a dentist would be able to discern liquid from solid material, or even clear fluids from infected (puss) fluids. One suitable material that can be manufactured to be substantially transparent or translucent is polypropylene.

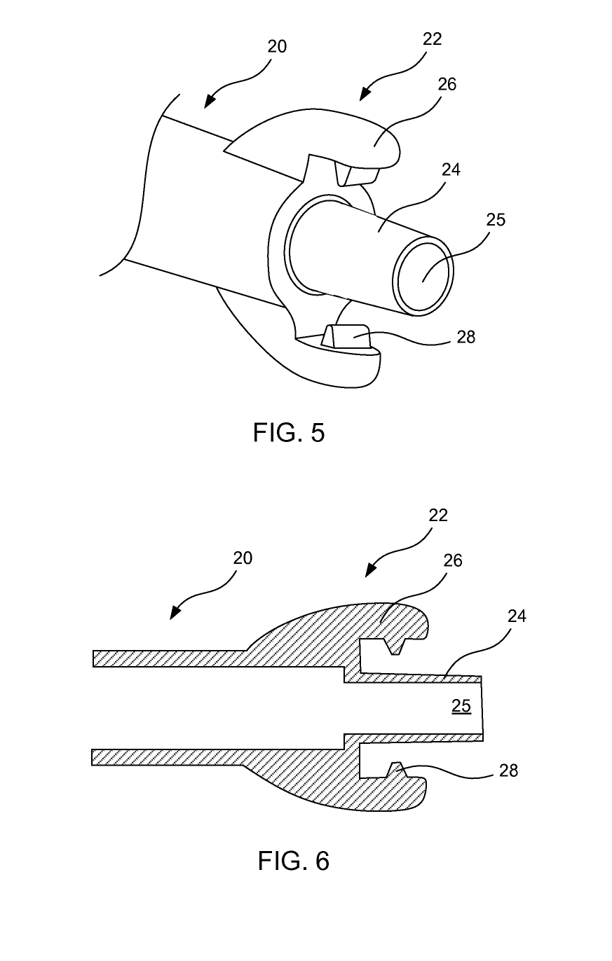

FIGS. 4 and 5 depict a perspective view and a cross-sectional view, respectively, of a male luer connector 22 that is present on a channeling body 20, e.g., suction tube, syringe barrel, flexible tubing, etc., in accordance with an example of the present disclosure. As shown, the male luer connector includes a male portion 25, a pair of arms 26, each having an engagement protrusion 28 extending inwardly thereof (toward the male portion). The male luer connector is positioned at a distal end or end of the suction tube about an opening 25.

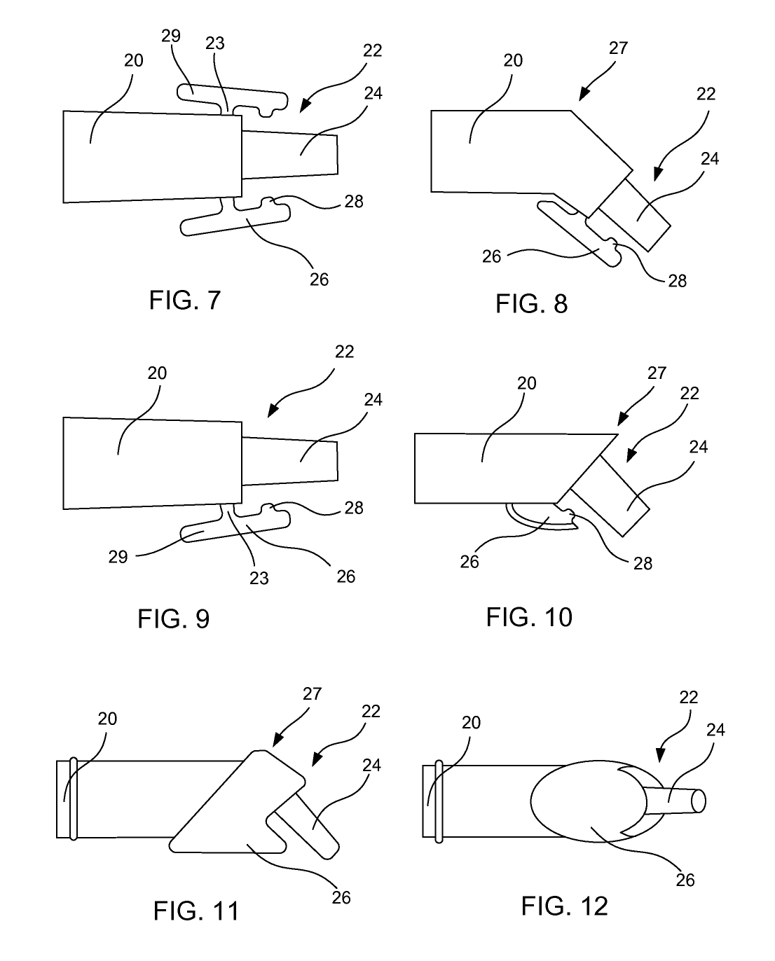

Turning now to FIGS. 7-12, various alternative examples are shown depicting various male luer connectors 22 at that are present at a distal end or end of a channeling body 20, such as a suction tube or syringe barrel, in accordance with examples of the present disclosure. Each of these examples includes a male portion 24 and an arm 26 (one arm as shown in FIGS. 8-12, or multiple arms as shown in FIG. 7). Furthermore, the male luer connectors of FIGS. 7-10 include an engagement protrusion 28, whereas the connector shown in FIGS. 11-12 does not include an extra engagement protrusion, relying more on a pressure fit to engage with the female luer adaptor of the adaptor tip. Furthermore, as shown specifically with respect to FIGS. 8, 10, 11, and 12, the male portion or the male luer connector is angled 27 with respect to the longitudinal axis of the channeling body, e.g., suction tube or syringe barrel. Furthermore, with specific reference to FIGS. 7-9, it is noted that the arm(s) include a lever 29 and a hinge 23, and the levers can be pressed toward the channeling body to provide a quick-release function for removing the adaptor tip (not shown).

While the forgoing examples and FIGS. are illustrative of the principles of the present technology in one or more particular applications, it will be apparent to those of ordinary skill in the art that numerous modifications in form, usage and details of implementation can be made without the exercise of inventive faculty, and without departing from the principles and concepts of the technology. Accordingly, it is not intended that the technology be limited, except as by the claims set forth below.

What is claimed is:

1. A device for injecting or extracting fluid, comprising:
a channeling body having a proximal end and a distal end, the channeling body configured for injecting or extracting fluid through an opening at the distal end; and
a male luer connector at the distal end about the opening, the male luer connector comprising an arm attached to the channeling body, wherein the male luer connector is adapted to engage with a female luer connector, and wherein the arm is also adapted to lock the female luer connector in place along an outer surface of the female luer connector.

2. The device of claim 1, wherein the channeling body is a suction tube configured for use with a suction extractor or ejector.

3. The device of claim 1, wherein the channeling body is a barrel of a syringe.

4. The device of claim 1, wherein the channeling body is includes flexible tubing.

5. The device of claim 1, wherein the male luer connector comprises two arms.

6. The device of claim 1, wherein the male luer connector consists of a single arm.

7. The device of claim 1, wherein the male luer connector consists of two arms.

8. The device of claim 1, wherein the arm further comprises an engagement protrusion protruding therefrom which is positioned and adapted to lock the female luer connector in place by engaging with a recess or a beyond a ridge on an outer portion of the female luer connector.

9. The device of claim 1, wherein the engagement protrusion is adapted to receive a threaded female luer connector.

10. The device of claim 1, wherein the arm comprises two engagement protrusion.

11. The device of claim 1, wherein the male luer connector comprises a first arm including a first engagement protrusion, and a second arm including a second engagement protrusion, wherein the first engagement protrusion on the first arm is misaligned with respect to the second engagement protrusion on the second arm.

12. The device of claim 1, wherein the male luer connector comprises a first arm including a first engagement protrusion, and a second arm including a second engagement protrusion, wherein the first engagement protrusion on the first arm is aligned with respect to the second engagement protrusion on the second arm.

13. The device of claim 1, wherein the male luer connector is angled with respect to the channeling body.

14. The device of claim 1, wherein the arm is hinged such that the arm can be opened and closed about the outer surface of the female luer connector.

15. The device of claim 1, wherein the device is a medical device.

16. A method of assembling a device for injecting or extracting fluid, comprising:
obtaining a channeling body having a proximal end and a distal end, the channeling body configured for injecting or extracting fluid through an opening at the distal end, wherein a male luer connector is also positioned at the distal end about the opening, and wherein the male luer connector comprises an arm attached to the channeling body;
obtaining an adaptor tip comprising a female luer connector; and
connecting the female luer connector to the male luer connector such that the arm locks the female luer connector in place along an outer surface of the female luer connector.

17. The method of claim 16, wherein the step of connecting is by twisting the adaptor tip onto the channeling body to lock the male luer connector and the female luer connector together.

18. The method of claim 16, wherein the step of connecting is by pushing a level of the arm over a hinged fulcrum, thereby creating enough space for a quick connection of the female luer connector with the shirtless male luer connector.

19. The method of claim 16, wherein the channeling body is a suction tube, a barrel of a syringe, or flexible tubing.

20. The method of claim 16, wherein the male luer connector comprises two arms.

21. The method of claim 16, wherein the arm further comprises an engagement protrusion protruding from the arm which is positioned and adapted to lock the female luer connector in place by engaging with a recess or beyond a ridge on an outer portion of the female luer connector.

22. The method of claim 16, wherein the male luer connector is angled with respect to the channeling body.

23. A modular system for injecting or extracting fluid, comprising:
an adaptor comprising a female luer connector; and
a channeling body having a proximal end and a distal end, the channeling body configured for injecting or extracting fluid through an opening at a distal end, the channeling body further comprising a male luer connector at the distal end about the opening, and wherein the male luer connector comprises an arm attached to the channeling body,
wherein the male luer connector is adapted to connect to the female luer connector such that the arm locks the female luer connector in place along an outer surface of the female luer connector.

24. The system of claim 23, wherein the channeling body is a suction tube configured for use with a suction extractor or ejector.

25. The system of claim 23, wherein the channeling body is a barrel of a syringe.

26. The system of claim 23, wherein the channeling body is includes flexible tubing.

27. The system of claim 23, wherein the male luer connector comprises two arms.

28. The system of claim 23, wherein the male luer connector consists of a single arm.

29. The system of claim 23, wherein the male luer connector consists of two arms.

30. The system of claim 23, wherein the arm further comprises an engagement protrusion protruding therefrom which is positioned and adapted to lock the female luer connector in place by engaging with a recess or beyond a ridge on an outer portion of the female luer connector.

31. The system of claim 23, wherein the engagement protrusion is adapted to receive a threaded female luer connector.

32. The system of claim 23, wherein the arm comprises two engagement protrusion.

33. The system of claim 23, wherein the male luer connector comprises a first arm including a first engagement protrusion, and a second arm including a second engagement protrusion, wherein the first engagement protrusion on the first arm is misaligned with respect to the second engagement protrusion on the second arm.

34. The system of claim 23, wherein the male luer connector comprises a first arm including a first engagement protrusion, and a second arm including a second engagement protrusion, wherein the first engagement protrusion on the first arm is aligned with respect to the second engagement protrusion on the second arm.

35. The system of claim 23, wherein the male luer connector is angled with respect to the channeling body.

36. The system of claim 23, wherein the arm is hinged such that the arm can be opened and closed about the outer surface of the female luer connector.

37. The system of claim 23, wherein the adaptor tip is a needle, a suction tip, a cap, a syringe adaptor, or a syringe.

38. The system of claim 23, wherein the adaptor tip is a suction tip having a opening at its working end of 0.005 inch to about 0.1 inch.

39. The system of claim 23, wherein the adaptor tip is a suction tip having a opening at its working end of 0.01 inch to about 0.05 inch.

40. The system of claim 23, wherein the channeling body and the adaptor comprise a medical device.
